(19) United States
(12) Patent Application Publication
(10) Pub. No.: US 2003/0229308 A1
(43) Pub. Date: Dec. 11, 2003

(54) Injector adapter and combination thereof

(76) Inventors: Israil Tsals, Newtown, PA (US); Gilad Lavi, Rishon Lezion (IL)

Correspondence Address: CAESAR, RIVISE, BERNSTEIN, COHEN & POKOTILOW, LTD., ATTN: ELAN, 12TH FLOOR, SEVEN PENN CENTER, 1635 MARKET STREET, PHILADELPHIA, PA, 19103-2212, US

(21) Appl. No.: 10/453,072

(22) Filed: Jun. 3, 2003

Related U.S. Application Data
(60) Provisional application No. 60/385,622, filed on Jun. 5, 2002.

(51) Int. Cl. A61M 5/00

(52) U.S. Cl. 604/116; 604/180; 604/506

(57) ABSTRACT

An injector adapter that permits an automatic injector to be releasably coupled to the skin of a living being without the need for a user to hold the injector against the skin during injection, especially in cases where the drug being delivered requires a slow rate of injection. In addition, another variation of the invention is described whereby the working end of the automatic injector includes an integrated injector adapter.

### RELATED APPLICATIONS

This application claims the benefit of U.S. Provisional Application No. 60/385,622 filed on Jun. 5, 2002 entitled INJECTOR ADAPTER AND COMBINATION THEREOF and whose entire disclosure is incorporated by reference herein.

### BACKGROUND OF THE INVENTION

The present invention relates to the administration of a drug solution and, more particularly, to the delivery of a viscous drug solution into a mammal.

Various devices have been developed for the delivery of medications into living organisms, including syringes in which a liquid is delivered from a chamber using pressure asserted by a manual plunger through a needle inserted under the skin. Improvements on these types of devices have led to needle-less injectors that apply a high pressure to the medication to create a jet that penetrates the skin without the need for a lumen.

However, the slowing down of injection rate is especially essential with the injection of viscous drug or other drugs that are difficult for tissue absorption. A level of pain usually follows injection due to the tissue's inability to quickly absorb the solution. Thus, a reduction of the injection rate is important for the injection of viscous drugs or other drugs that are difficult for tissue absorption because, in many cases, pain is the result of rate differences between the injection rate and the absorption rate. Other reasons for the reduced injection rate include the prevention of an adverse reaction due to rapid compound injection and the provision for lower injection forces needed for viscous compounds.

Where it is necessary to slow down the injection rate of an injector, it also necessary to hold and stabilize the injector in place during the injection process. This is not always easy to do and in fact can lead to increased pain when the attendant or patient tries to hold the injector steady but unconsciously or inadvertently moves the injector slightly during drug delivery. In addition, some of the drugs used by patients are for patients having limited dexterity. These patients are incapable of holding an injector in a stable orientation for a prolonged period of time. Thus, having to hold the injector throughout the injection process (e.g., 10 minutes) can be not simply tedious, but can be impossible for some patients. Moreover, having to hold the injector for a prolonged period of time can increase the pain to the patient as a result of movement. In addition, if a health care professional is administering, it diverts their attention from other tasks such as monitoring the patient's vital signs and/or taking appropriate actions (operating nearby equipment) necessary to stabilize/maintain the patient's health.

The prior art provides no solution to this problem. In the broad field of injection devices, the prior art does disclose needle “disposing” devices, such as those discussed in British Application No. 2205043, U.S. Pat. Nos. 4,836,373 (Goldman), 4,717,386 (Simmons); 4,737,149 (Gillian); 4,915,698 (Levenson); 5,334,173 (Armstrong et al.); 5,078,696 (Nedbaluk); 5,505,705 (Galpin et al.); 6,202,843 (Kelson et al.)); and needle/catheter “guiding” devices, such as those shown in U.S. Pat. Nos. 5,348,543 (Talley); 2,402,306 (Turkel); 2,295,849 (Kayden); 3,900,026 (Wagner); 4,755,173 (Konopa et al.); 4,318,401 (Zimmerman); 5,545,143 (Fischell)); 5,496,264 (Watson et al.); needle sheathing/handling devices, such as those shown in British Patent Application No.2205043 (Jones et al.) British Patent Application No. GB 2209470 (Calvert et al.); French Patent Application No. 2635686 (Fochesato et al.); and International Publication WO 91/00215 (Melker et al.); and needle guides for use with infusion devices such as that shown in U.S. Pat. No. 4,675,006 (Hrushesky).

PCT International Patent No. WO 96/24398 (Weston) discloses a needle-less injector that utilizes a pressure pad for evenly distributing the contact pressure of the injector when it is placed on the skin but also does not teach or suggest supporting the needle-less injector on its own for a prolonged period of time.

U.S. Pat. No. 6,332,875 (Inkpen et al.) discloses a hand-held injection device that facilitates the injection of manually-operated syringes or pens.

Thus, there remains a need for a device that permits an automatic injector to be applied to an injection site on a living being which can support itself for a prolonged period of time and automatically deliver a drug.

### SUMMARY OF THE INVENTION

An apparatus for use with an automatic injector (e.g., any injector that automatically delivers a solution to a living being including automatic syringes, automatic needle-less injectors, etc.,) for injecting a solution into a living being. The apparatus comprises: an injector-receiving member (e.g., a collar or sleeve) for receiving a portion of a housing of the automatic injector; and a living being-engaging member (e.g., a substantially flat base surface) forming one end of the injector-receiving member and including releasable coupling means (e.g., a biocompatible adhesive layer) for releasably coupling the apparatus to the living being, and wherein the living being-engaging member comprises an aperture for permitting passage of the solution from the automatic injector, when activated, into the skin of the living being.

A method for delivering a solution to a living being using an automatic injector (e.g., any injector that automatically delivers a solution to a living being including automatic syringes, automatic needle-less injectors, etc.,) having an output for dispensing the solution. The method comprises the steps of: coupling a rigid structure (e.g., a substantially flat base portion having a collar or sleeve member forming a part thereof) to the skin of the living being, and wherein the rigid structure has an aperture in a portion thereof (e.g., in the substantially flat base portion) that couples to the skin of the living being; coupling the automatic injector to the rigid structure; and activating the automatic injector to cause the solution to pass from the output, through the aperture and into the skin.

An apparatus for automatically delivering a solution to a living being. The apparatus comprises: an automatic injector (e.g., any injector that automatically delivers a solution to a living being including automatic syringes, automatic needle-less injectors, etc.,) having a housing with a proximal and distal end, wherein the proximal end comprises a skin-engaging surface and a hole for permitting the solution to pass therethrough; the skin-engaging surface comprises releasable coupling means for releasably coupling the apparatus to the living being; and wherein the apparatus delivers the solution, when activated, through the skin of the living being without the need for a person to hold the apparatus during solution delivery.

A method for automatically injecting a solution into a living being. The method comprising the steps of: coupling a proximal end of an automatic injector (e.g., any injector that automatically delivers a solution to a living being including automatic syringes, automatic needle-less injectors, etc.,) to the skin of the living being, wherein the automatic injector has an aperture in the proximal end that is coupled to the skin of the living being; and activating the automatic injector to cause the solution to pass through the hole and into the skin and without the need for a person to hold the automatic injector during solution delivery.

### BRIEF DESCRIPTION OF THE DRAWINGS

FIG. 1 is an isometric view of the injector adapter of the present invention;

FIG. 2 is an isometric view of the injector adapter including an exploded view of a preferred adhesion system that is coupled to the base portion of the injector adapter for releasably coupling the injector adapter to the skin of a living being;

FIG. 2A is an isometric view of the preferred adhesion system of FIG. 2;

FIG. 3 is an exploded view showing an automatic injector and the injector adapter, with the injector adapter already applied to the skin of a living being;

FIG. 4 depicts the automatic injector engaged with injector adapter, shown in cross-section, and with the automatic injector being activated to make the injection into the skin;

FIG. 5 depicts the automatic injector engaged with another embodiment of the injector adapter having a shortened injector-receiving portion, shown in cross-section, and with the automatic injector being activated to make the injection into the skin;

FIG. 6 is an isometric view of another embodiment of the injector adapter using the shortened injector-receiving portion, showing a release sheet tab projecting from the base of the adapter;

FIG. 7 is an exploded view showing another automatic injector and the injector adapter of FIG. 6 before the adapter is applied to the skin;

FIG. 8 depicts the automatic injector engaged with injector adapter of FIG. 6 but with the release sheet still covering the base of the adapter;

FIG. 9 depicts a bottom isometric view of the injector adapter using the shortened injector-receiving portion of FIGS. 5-8 and including the preferred adhesion system;

FIG. 10 is a side elevation view of an integrated injector adapter of the present invention;

FIG. 11 is a top view of the integrated injector adapter;

FIG. 12 is a partial cross-sectional view of the lower portion of the integrated injector adapter taken along line 12-12 of FIG. 11;

FIG. 13a is a top view of the clip of the integrated injector adapter;

FIG. 13b is a bottom view of the clip the integrated injector adapter;

FIG. 14 is a partial cross-sectional view of one snap of the clip engaged with the body-engaging portion of the integrated injector adapter taken along line 14-14 of FIG. 13a;

FIG. 15 is a top view of the release liner of the integrated injector adapter; and

FIG. 16 is a partial isometric and cross-sectional view of the upper portion of the clip of the integrated injector adaptor illustrating how a tab of the release liner is secured to the clip.

### DETAILED DESCRIPTION OF THE INVENTION

As discussed earlier, reduction of the injection rate is important for the injection of viscose drugs or other drugs that are difficult for tissue absorption because, in many cases, pain is the result of rate differences between the injection rate and the absorption rate. Thus, as will be discussed below in detail, the injector adapter 20 permits the automatic injector to be attached to the skin of the living being, without the need for the patient or an attendant (hereinafter “user”) to hold the injector, for the required duration of the slow injection. Furthermore, it is within the broadest scope of this invention that the injector adapter 20 be universal. That is, the injector adapter 20 can be applied to a range of conventional automatic injectors where the delivery of a highly viscous drug is required, and/or where the time of drug delivery is longer than a conventional automatic injector delivering a bolus and/or where manual injection by the patient is not possible and assistance is needed in the form of the adapter 20. The high viscosity of some drug solutions substantially increases the injection time. Injection time is the result of the level of pressure applied to the drug volume, the physical attributes of the drug including viscosity, and the fluid path geometry such as the needle's inner diameter and length, as well as delivery volume. Injection time, in most cases, is a device control parameter though in some cases the tissue also affects it by creating significant backpressure. Alternatively, for some low viscosity drugs, a modified device would be used to slow down the delivery rate and to increase the delivery time. The injection time could reach into tens of minutes. It is not possible for the patient to reliably keep an injector pressed against the skin for an extended time. The injector adapter 20 solves this problem.

Referring now in detail to the various figures of the application wherein like reference characters refer to like parts, there is shown at 20 in FIG. 1 an injector adapter that couples to the skin of a living being and permits an automatic injector to be coupled thereto without the need for an attendant to hold the automatic injector during drug delivery to the living being; especially in cases where the injection of the drug solution must be delivered slowly over a long period of time (e.g., approximately 10 minutes).

In particular, the injector adapter 20 basically comprises a base portion 22 and an injector receiving portion 24. As shown most clearly in FIG. 3, the base portion 22 couples to the skin 26 of a living being and an automatic injector 10 is releasably coupled to the injector receiving portion 24. Examples of automatic injectors that can be used with the injector adapter 20 are those disclosed in U.S.A.S. No. 60/334,294 filed on Nov. 30, 2001 entitled AUTOMATIC INJECTOR and whose entire disclosure is incorporated by reference herein. It should be understood that it is within the broadest scope of this invention to have the injector adapter 20 be used with any automatic injector and that the examples cited in No. 60/334,294 do not limit the use of the injector adapter 20 to only those automatic injectors. Moreover, the term “automatic injector” as used throughout this Specification is meant to include any injector that automatically delivers a material to a living being and therefore includes automatic injectors that do not automatically retract an injection needle (also referred to as “automatic syringes”) and also includes automatic needle-less injectors.

The preferred embodiment of the injector adapter 20 comprises a unitized base portion 22 and injector receiving portion 24 that can be formed using any well-known rigid material (e.g., plastic), although it is within the broadest scope of the invention 20 to have the base portion 22 and the injector-receiving portion 24 be two separate pieces that can be assembled into the injector adapter 20. The injector adapter 20 forms a disposable unit, as do the automatic injectors that are used with the injector adapter 20.

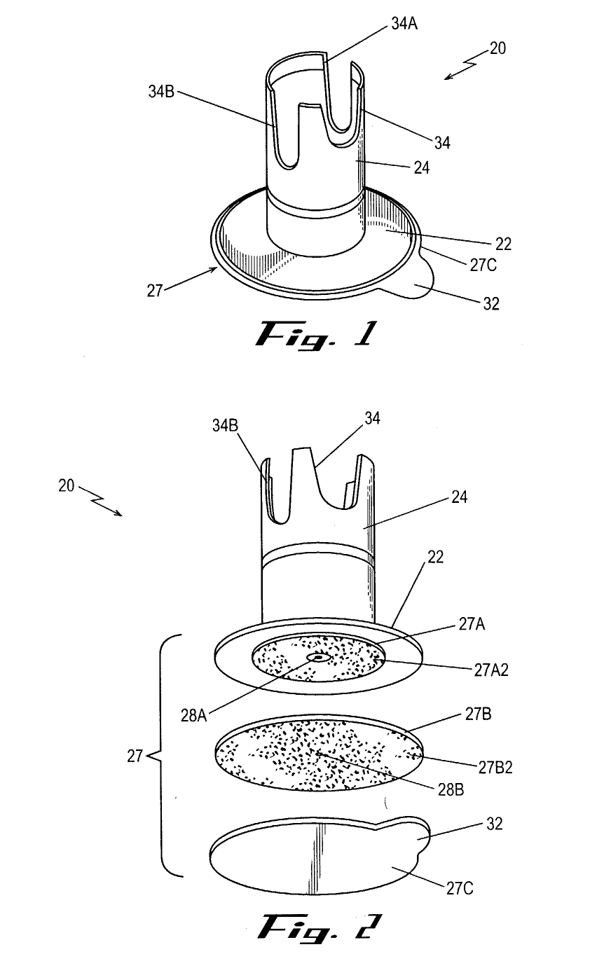

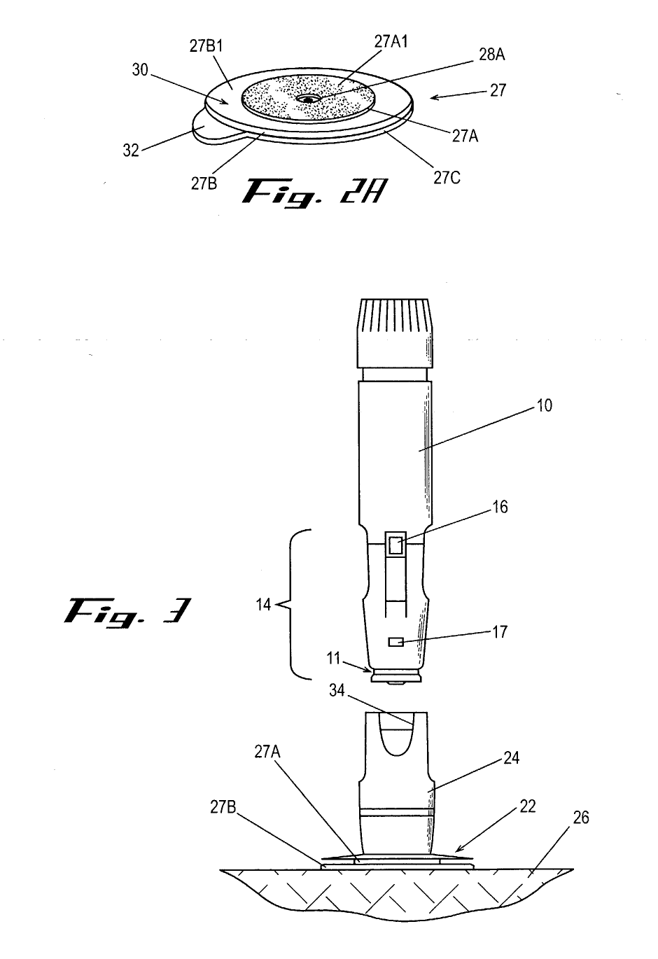

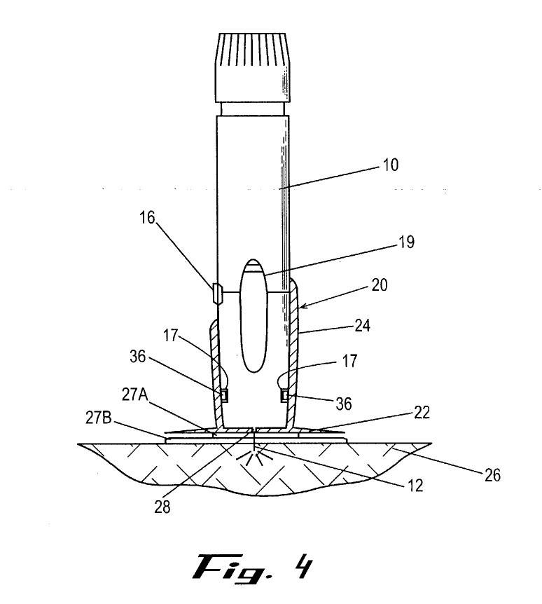

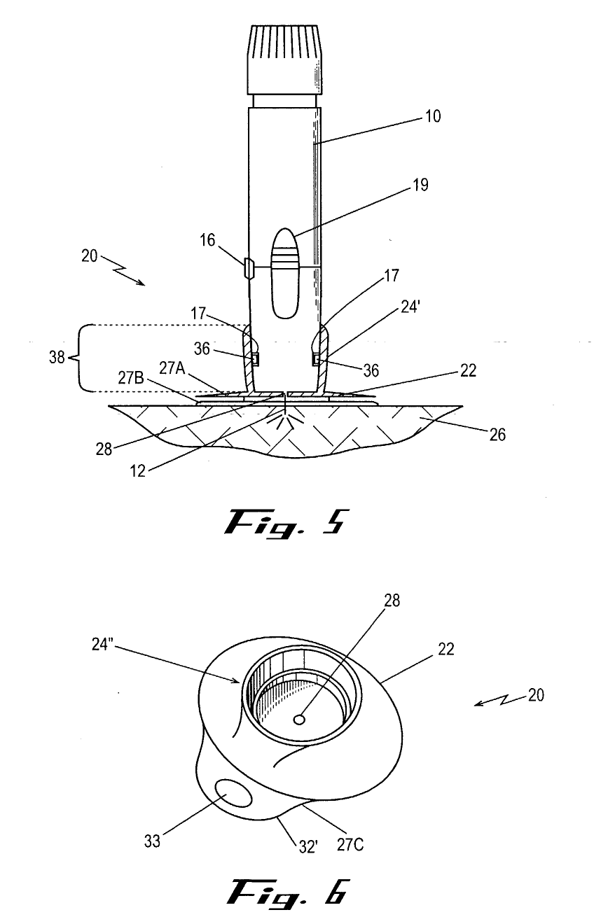

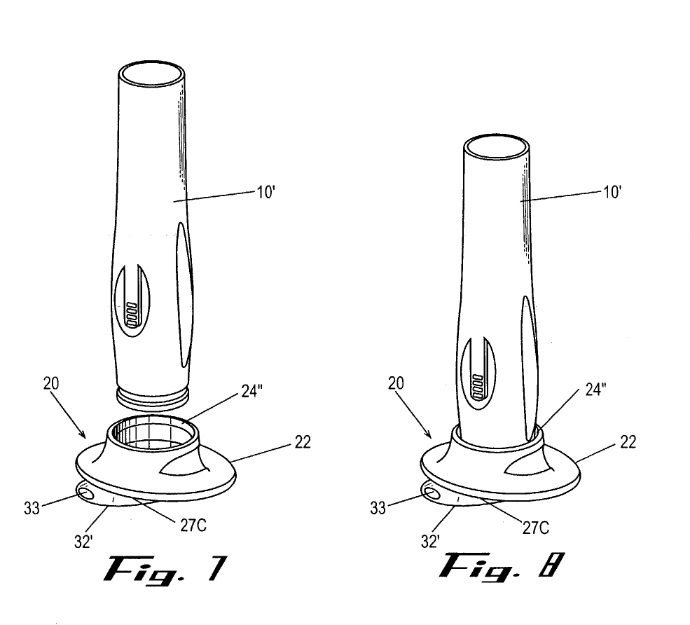

The base portion 22 forms a substantially flat surface and comprises a hole 28 (FIGS. 4-5) that permits a needle 12 (FIG. 4) to pass through the base portion 22 and into the skin 26 for the injection; alternatively, when a needle-less automatic injector (not shown) is used, the hole 28 permits the passage of the drug solution from the output of the needle-less automatic injector, through the base portion 22 and into the skin 26. The base portion 22 is circular in shape (e.g., 60 mm diameter) and, in the preferred embodiment, adheres to the skin 26 of the living being using, preferably, an adhesive system 27 (FIGS. 2-2A) applied to the underside of the base portion 22 to maintain the automatic injector 10 pressed against the skin 26 of the living being for an extended time.

In order to couple the injector adapter 20 to the skin 26, it is necessary to allow an element of flexibility for the skin 26 relative to the rigid nature of the injector adapter 20 and injector 10. A similar adhesive mechanism has been developed and is the subject of PCT Application No. IE99/00046 entitled IMPROVED ADHESIVE SYSTEM FOR MEDICAL DEVICES and whose entire disclosure is incorporated by reference herein. The adhesive system 27 improves the ability to attach a rigid body, i.e., the injector adapter 20, to a flexible one, i.e., the skin 26 of a living being. In particular, as shown in FIG. 2, the adhesive system 27 comprises a double-sided adhesion layer 27A, a biocompatible single-side adhesion layer 27B and a liner 27C. The double-sided adhesion layer 27A (e.g., Avery Dennison FT-1115) has one side 27A1 (FIG. 2A) that is secured against the base portion 22; a hole 28A is provided in the layer 27A that aligns with the hole 28 in the base portion 22 for allowing passage of the drug or needle (not shown). The non-adhesive side 27B1 (a portion of which is shown in FIG. 2A) of the biocompatible single-side adhesion layer 27B (e.g., Kendall FM100) is secured against the other side 27A2 of the double-sided adhesion layer 27A; similarly, the biocompatible single-side adhesion layer 27B also comprises a hole 28B that aligns with holes 28/28A to permit passage of the drug or needle (not shown). Finally, the liner 27C having a pull tab 32 is releasably coupled to biocompatible adhesive side 27B2 to cover side 27B2 until that side 27B2 is ready to be coupled to the skin 26. As can be seen from FIGS. 2-2A, the biocompatible single-side adhesion layer 27B is larger than the double-sided adhesion layer 27A, thereby forming a “skirt” or “ring” 30 that is not adhered to the base portion 22. As a result, this provides the element of flexibility mentioned earlier. During assembly, the adhesive system 27 (FIG. 2A) is applied to the base portion 22 and is ready for use as shown in FIG. 1. When the injector adapter 20 is ready to be applied, the user seizes the tab 32 on the liner 27C and pulls it away from the base portion 22, thereby exposing the biocompatible adhesive side 27B2. Once the liner 27C is removed, the injector adapter 20 can then be adhered to the injection site on the skin 26. Tests conducted using the adhesive system 27 with the injector adapter 20 with an automatic injector 10 coupled thereto indicated that it is important to thoroughly wipe the injection site with alcohol before the injector adapter 20 is applied to the area. This wiping procedure apparently reduces the levels of oil and particulate matter in the site area which enhances the ability of the biocompatible adhesive side 27B2 to hold the injector adapter 20 in place for a long period of time (e.g., 10 minutes).

The injector receiving portion 24 of the injector adapter 20, in the preferred embodiment, basically comprises a collar or neck that receives the working end 14 of the automatic injector 10. In particular, the collar/sleeve is circular in shape (e.g., inside diameter 24 mm) in order to correspond with the circular shaped working end 14 of the injector 10. It should be noted that the collar/sleeve may be a non-circular shape (e.g., oval) so long as it corresponds in shape with the working end of the injector 10. Depending on the type of automatic injector 10 used, it is within the broadest scope of the injector adapter 20 to have the injector receiving portion 24 include means for engaging and disengaging. For example, if the injector adapter 20 is to be a non-disposable unit and therefore used again, disengagement from the automatic injector 10 is then required. Thus, the working end 14 of the injector 10 may include tabs, undercuts, and cutouts in the collar/sleeve to permit unobstructed operation of any injector triggers or visual access to any drug windows when the injector 10 is engaged with the injector adapter 20. Thus, the injector adapter 20 can have design features that match a selected automatic injector design.

For example, as shown most clearly in FIG. 3, where an automatic injector 10 comprises a slidable injection trigger 16 along the working end 14, the injector receiving portion 24 comprises a cutout 34 (or opening, aperture, gap, etc.) to allow the attendant to operate the trigger 16 once the working end 14 is coupled to the injector adapter 20. FIG. 4 shows the trigger 16 being in a downward position, thereby extending the needle 12 downward, through the hole 28 and into the skin 26. Furthermore, where the automatic injector 10 comprises a slit/cavity 17 (see FIGS. 3-4) at the working end 14, a corresponding catch 36 (FIG. 4) inside the collar/sleeve releasably engages the slit/cavity 17 to releasably couple the injector 10 to the injector adapter 20. In addition, where a drug window 19 (FIGS. 4-5) is included within the working end 14 of the injector 10, a corresponding cutout 34A (FIG. 1, or other cutout, opening, aperture, gap, etc.) in the injector receiving portion 24 provides visual access to the user during the injection process to permit the user to know the level of drug still within the injector 10; the injector receiving portion 24 may include other cutouts 34B (FIG. 1) that permit visual access to other portions of the working end 14 of the injector 10 when it is engaged in the injector receiving portion 24. It should also be noted that where the automatic injector 10 comprises a body sensing element 11 that normally retracts when the injector 10 is directly pressed against the skin of the living being, this retraction is not obstructed when the injector 10 is coupled to the injector adapter 20. In particular, during engagement of the working end 14 of the injector 10 within the injector adapter 20, the base portion 22 provides the body sensing element 11 with a firm surface to push against; in addition, this retraction action of the body element 11 operates to cause a snap cooperation of the slit/cavity 17 and the catches 36 to support the engagement/disengagement of the working end 14 within the injector receiving portion 24. Furthermore, this snap cooperation also aligns the retracted injection needle 12 with the hole 28 (as well as holes 28A-28B in the adhesion system 27) in preparation for injection. Once engaged with the injector adapter 20, it should be understood that the automatic injector 10 can then be used in its normal operation (i.e., needle penetration, drug delivery, needle retraction by the end of delivery) as if it were placed directly against the skin, but without the need to hold the injector 10 in place during injection.

It should be noted that although it is preferred to have the automatic injector 10 be vertically oriented during the injection process (as shown in FIG. 4), e.g., the living being can be in a prone position, it is not required. If the living being is a human, the patient can be in a sitting, reclining or even in an upright position. Furthermore, the preferred embodiment of the injector adapter 20 has the injector receiving portion 24 perpendicular to the base portion 22, thereby creating an injection that is normal to the skin surface. It is within the broadest scope of the invention 20 though, to include other angular orientations of the injection to the skin using the injector adapter 20. Thus, the key feature of the present invention 20 is that it provides a secure coupling of the automatic injector 10 to the living being during injection without the need for an attendant to hold the injector 10 during injection, thereby preventing any needle movements in the tissue during injection.

An alternative embodiment of the injector adapter 20 is shown in FIG. 5 whereby the injector receiving portion 24′ of the injector adapter 20 is shortened in height. This shortened height 38 facilitates packaging and storing of the injector adapter 20. It also alleviates the need to provide a cutout 34 (or opening, aperture, gap, etc.) in the collar/sleeve as discussed earlier. And although the neck/collar is shortened, the injector-receiving portion 24 still operates to couple a smaller sized working end of the injector 10 using the snap cooperation of the slit/cavity 17 and the catches 36. FIGS. 6-8 show the use of the injector adapter 20 using another type of shortened injector receiving portion 24″ with a different type of automatic injector 10′. The release sheet 27C also comprises a slightly-modified tab 32′ which includes a grasping hole 33. FIG. 9 shows the injector adapter 20 using either of the shortened injector receiving portions 24′ or 24″ and the adhesive system 27 coupled to the base portion 22, with the liner 27C already removed.

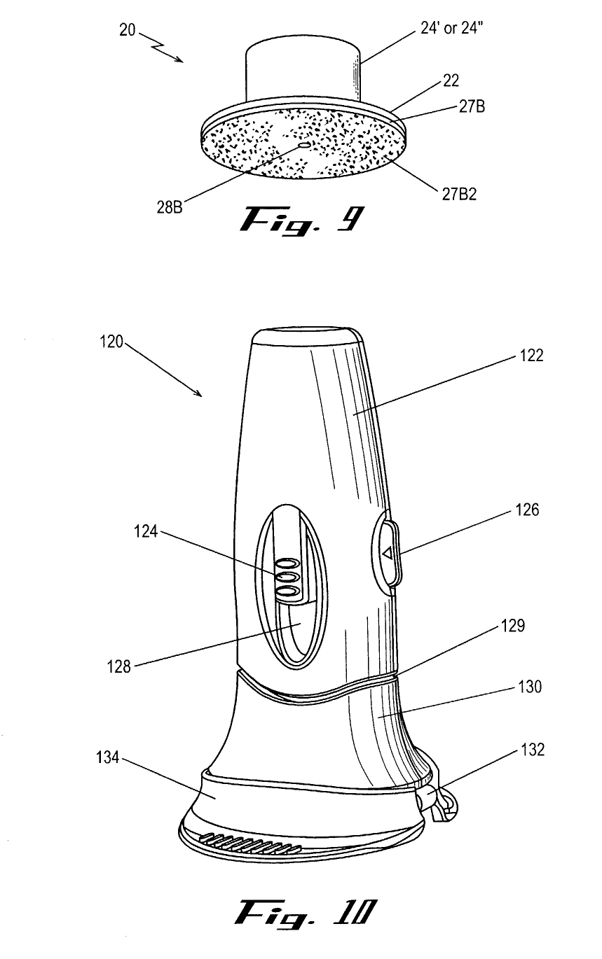

Another variation of the injector adapter 20 is an integrated injector/adapter configuration as shown in FIG. 10 using the reference number 120, whereby the adapter is incorporated into the automatic injector itself at its proximal end. The integrated injector adapter 120 reduces the number of steps and simplifies the injection process. This is of critical importance for patients with limited dexterity, including rheumatoid arthritis (RA) and multiple sclerosis (MS) patients. In the embodiments of the injector adapter 20 discussed previously, the user would remove the injector adapter 20 from its package and the automatic injector 10 from its package. The user would then normally apply the injector adapter 20 to the injection site (after thoroughly wiping the site with alcohol, as discussed earlier) by pulling the tab 32 of the release sheet 27C and applying the injector adapter 20 at the site. Next, the user would prepare the automatic injector 10 in accordance with the accompanying instructions of that particular injector 10 (e.g., inserting a drug cartridge, removing any needle protector means, etc.). Once the injector 10 is ready for use, the user would then insert the working end 14 of the injector 10, as discussed previously. In contrast, using the integrated injector adapter 120, the user would remove the combined automatic injector/adapter 120 from a single package. Furthermore, the removal of the release sheet 27C from the base portion 22 of the integrated injector adapter 120 simultaneously removes the needle protector means. Because the automatic injector 10 already comprises the base portion 22, the step of inserting the automatic injector 10 into the adapter is omitted.

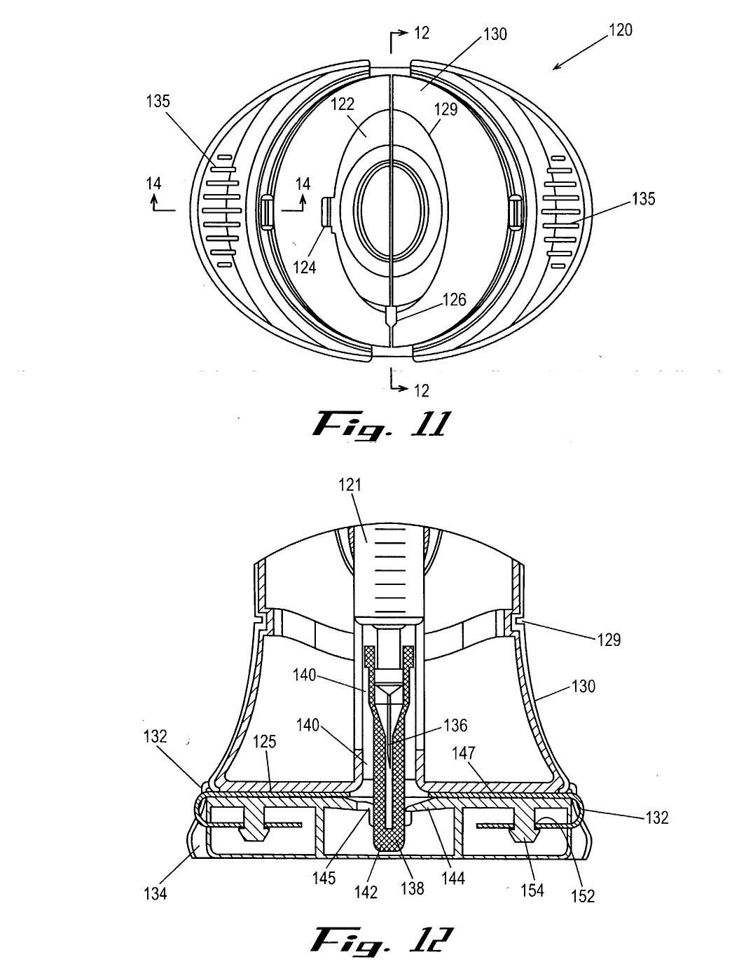

As shown most clearly in FIGS. 10-11, the integrated injector adapter 120 comprises an injector 122, a trigger button 124, a safety tab 126, an observation window 128, the body-engaging portion 130, a liner 132 and a clip 134. A form or bond line 129 is shown between the injector 122 housing and the body-engaging portion 130. As will be discussed in detail later, the clip 134 is pulled away from the body-engaging portion 130 when the device 120 is ready to be applied to the skin 126 of the living being. When the clip 134 is pulled away, a flat base surface 125 (FIG. 12) is exposed for engaging the skin 126 of the living being.

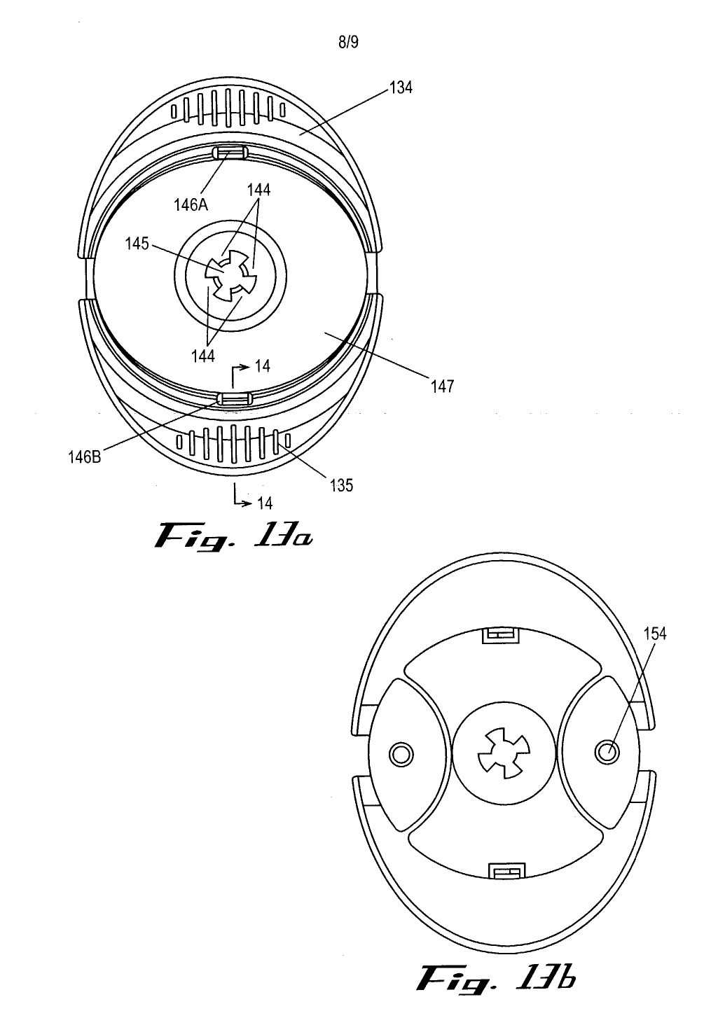

In particular, as shown most clearly in FIG. 12, the injector 122 contains a pre-filled syringe 121 (e.g., a pre-filled HYPAK® syringe, such as a 1 ml “short” version),having a needle 136 attached thereto and a removable needle protector 138 (e.g., a rubber sterility protector) covering the needle 136. When the integrated injector adapter 120 is assembled, the needle 136 and protector 138 are positioned to pass through an opening 140 in the flat base surface 125. The forward end 142 of the needle protector 138 is lodged into latches 144 located within an opening 145 (FIG. 13a) in the top surface 147 of the clip 134. Thus, upon removal of the clip 134, the needle protector 138 is also pulled away from the needle 136, while the needle 136 remains retracted within the body-engaging portion 130, in preparation for the injection.

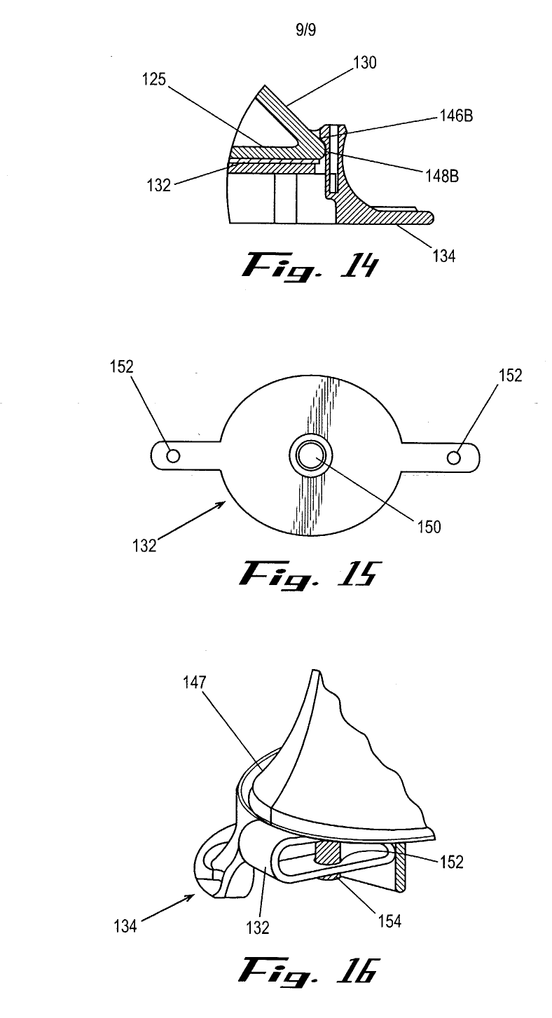

FIG. 13a provides a top view of the clip 134 (removed from the integrated injector adapter 120) showing the opening 145 containing the latches 144 therein. A pair of snaps 146A/146B on opposing sides of the clip 134 releasably engage corresponding recesses in the lower portion of the body engaging portion 130, as shown most clearly in FIG. 14 which shows only one of the snaps 146B/corresponding recess 148B, it being understood that the other snap 146A releasably engages into a corresponding recess on the opposite side of the body engaging portion 130. The clip 134 has a gripping surface 135 (FIG. 13a) which helps an RA or MS patient seize the clip 134 in order to remove it.

FIG. 15 provides a top view of the release liner 132 which covers the flat base surface 125 before use of the integrated injector adapter 120. During assembly of the integrated injector adapter 120, an adhesive system, similar to adhesive system 27, is applied to the underside surface of the flattened base surface 125 and includes a biocompatible single-side adhesion layer (similar to layer 27B discussed earlier) that is covered with the release liner 132. A hole 150 is provided in the release liner 132 to permit the removal of the needle protector 138 when the clip 134 is pulled away. In addition, the release liner 132 comprises holes 152 on opposite sides of the release liner 132 which are connected to clip 134 via respective pins 154 (only one of which is shown in FIG. 16) which are on opposite sides of the clip 134. Thus, when the user pulls the clip 134 away from the integrated injector adapter 120, the release liner 132 is also pulled away in a symmetrical manner that will not cause any side forces on the protector 138 or on the needle 136 that might otherwise bend or deform either the protector 138 or the needle 136. This removal of the clip 134 exposes the adhesion biocompatible single-side adhesion layer 27B2 in preparation for application of the integrated injector adapter 120 to the skin 26.

During assembly, the pre-filled syringe 121, including the needle 136 and needle protector 138, is positioned within the injector 122 housing and positioned through the hole 140 in the body-engaging portion 130. The release liner 132 is installed on the flat base surface 125 and the clip 134 is coupled to the body-engaging portion 130. When the integrated injector adapter 120 is to be used for injection, the user wipes the injection site thoroughly with alcohol. The user then seizes the gripping surface 135 of the clip 134 and pulls the clip 134 away from the integrated injector adapter 120, which exposes the biocompatible single-side adhesion layer (similar to layer 27B discussed earlier). The user then presses the flat base surface 125 against the injection site to cause the integrated injector adapter 120 to adhere to the injection site. The user then removes the safety tab 126; the safety tab 126 prevents false activation of the integrated injector adapter 120. As discussed earlier with respect to the injector adapter 20, some automatic injectors 10 use a body-sensing element 11 (FIG. 3) as a method to prevent false activation. Where the integrated injector adapter 120 is used, such a body-sensing element is replaced by the safety tab 126. Next, the user then activates the trigger 124 to effect the automatic injection: needle penetration and drug delivery for the prolonged period (e.g., 10 minutes) which is then followed by automatic needle retraction. The user can also confirm completion of the drug delivery by sensing the automatic needle retraction and/or viewing the empty syringe body which is visible through the drug window 128. The integrated injector adapter 120 is then removed from the injection site and ready for safe disposal.

It should be understood that both the injector adapter 20 and the integrated injector adapter 120 can be used for extended bolus as well as by patients not capable of keeping the automatic injector steady against the injection site for the time required for conventional automatic injections (e.g., a few seconds).

It is also within the broadest scope of the invention to include the use of an injector adapter for conventional hypodermic needles that are manually-operated by an attendant.

It is further appreciated that the present invention may be used to deliver a number of drugs. The term “drug” used herein includes but is not limited to peptides or proteins (and mimetic thereof), antigens, vaccines, hormones, analgesics, anti-migraine agents, anti-coagulant agents, medications directed to the treatment of diseases and conditions of the central nervous system, narcotic antagonists, immunosuppressants, agents used in the treatment of AIDS, chelating agents, anti-anginal agents, chemotherapy agents, sedatives, anti-neoplastics, prostaglandins, antidiuretic agents and DNA or DNA/RNA molecules to support gene therapy.

Typical drugs include peptides, proteins or hormones (or any mimetic or analogues or any thereof) such as insulin, calcitonin, calcitonin gene regulating protein, atrial natriuretic protein, colony stimulating factor, betaseron, erythropoietin (EPO), interferons such as α, β or γ interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovacular agents, anti-hypertensive agents and vasodilators such as diltiazem, clonidine, nifedipine, verapamil, isosorbide-5-monotritate, organic nitrates, agents used in treatment of heart disorders, and analogues thereof; sedatives such as benzodiazepines, phenothiazines, and analogues thereof; chelating agents such as defroxanune, and analogues thereof; anti-diuretic agents such as desmopressin, vasopressin, and analogues thereof; anti-anginal agents such as fluorouracil, bleomycin, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluvoxamine, bisoprolol, tacrolimus, sacrolimus and cyclosporin.

While this invention has been particularly shown and described with references to preferred embodiments thereof, it will be understood by those skilled in the art that various changes in form and details may be made therein without departing from the spirit and scope of the invention as defined by the appended claims.

We claim:

1. An apparatus for use with an automatic injector for injecting a solution into a living being, said apparatus comprising:
an injector-receiving member for receiving a portion of a housing of the automatic injector; and
a living being-engaging member forming one end of said injector-receiving member and including releasable coupling means for releasably coupling said apparatus to the living being, said living being-engaging member comprising an aperture for permitting passage of the solution from said automatic injector, when activated, into the skin of the living being.

2. The apparatus of claim 1 wherein said automatic injector delivers the solution, when activated, without the need for a person to hold said apparatus during solution delivery.

3. The apparatus of claim 2 wherein said injector-receiving member and said living being-engaging member form a unitized structure.

4. The apparatus of claim 2 wherein said injector-receiving member comprises a longitudinal axis and wherein said living being-engaging member is oriented perpendicular to said longitudinal axis.

5. The apparatus of claim 2 wherein the automatic injector includes a needle and wherein said aperture permits the passage of said needle therethrough and into the skin to deliver the solution to the living being.

6. The apparatus of claim 2 wherein said releasable coupling means comprises an adhesive means coupled to said living being-engaging member.

7. The apparatus of claim 6 wherein said adhesive means comprises:
a first layer comprising a first size and having a first pair of opposing surfaces and wherein one of said first pair of opposing surfaces is coupled to said living being engaging member; and
a second layer comprising a second size larger than said first size and including a second pair of opposing surfaces, wherein one of said second pair of opposing surfaces is coupled to the other one of said first pair of opposing surfaces and wherein the other one of said second pair of opposing sides comprises a biocompatible adhesive.

8. The apparatus of claim 7 wherein said one said first pair of opposing surfaces comprises an adhesive such that said first layer is adhesively secured to said living being-engaging member.

9. The apparatus of claim 8 wherein the other one of said first pair of opposing surfaces also comprises an adhesive such that said one of said second pair of opposing surfaces is also adhesively coupled to the other one of said first pair of opposing surfaces.

10. The apparatus of claim 8 further comprising a release liner that is releasably coupled to the other one of said second pair of opposing sides which compromises said biocompatible adhesive.

11. The apparatus of claim 2 wherein said injector receiving member comprises a collar into which said portion of the automatic injector housing that includes said needle is fitted.

12. The apparatus of claim 11 wherein said housing of said automatic injector comprises an operating trigger, said collar comprising a cutout to permit the unobstructed operation of said trigger.

13. The apparatus of claim 11 wherein said collar comprises a releasable-engaging means that cooperates with a corresponding releasable engaging means located on said portion of the automatic injector housing that includes said needle.

14. The apparatus of claim 2 wherein said releasable coupling means comprises a fabric having a first side that is fixedly secured to said living being-engaging member and a second side that comprises an adhesive for contact with the skin of the living being.

15. The apparatus of claim 11 wherein said adhesive comprises a biocompatible adhesion layer.

16. The apparatus of claim 5 wherein said needle passes through said aperture and into the skin of the living being for a predetermined period of time and wherein said predetermined period of time comprises at least 10 minutes.

17. A method for delivering a solution to a living being using an automatic injector having an output for dispensing the solution, said method comprising the steps of:
coupling a rigid structure to the skin of the living being, said rigid structure having an aperture in a portion thereof that couples to the skin of the living being;
coupling said automatic injector to said rigid structure; and
activating said automatic injector to cause the solution to pass from the output, through said aperture and into said skin.

18. The method of claim 17 wherein said step of activating said automatic injector does not require a person to hold said rigid structure during solution delivery.

19. The method of claim 18 wherein said step of coupling a rigid structure to the skin of the living being comprises:
applying an adhesive to said portion of said rigid structure that eventually is in contact with the skin of the living being;
covering said adhesive with a release liner;
wiping the skin of the living being where said rigid structure is to be coupled;
removing said release liner from said portion of said rigid structure when ready for use; and
applying firmly said portion of said rigid structure to the skin of the living being.

20. The method of claim 19 wherein said step of applying an adhesive to said portion of a rigid structure comprises:
coupling one surface of a first pair of opposing surfaces of a first layer, having a first size, to said portion of said rigid structure;
coupling one surface of a second pair of opposing surfaces of a second layer having a second size, larger than said first size, to the other one of said first pair of opposing surfaces; and
covering the other one of said second pair of opposing surfaces with a biocompatible adhesive.

21. The method of claim 19 wherein said step of coupling one surface of a first pair of opposing surfaces of a first layer to said portion of said rigid structure comprises applying an adhesive to said one surface of said first pair of opposing surfaces.

22. The method of claim 21 wherein said step of coupling one surface of a second pair of opposing surfaces of a second layer having a second size, to the other one of said first pair of opposing surfaces comprises applying an adhesive to the other one of said first pair of opposing surfaces.

23. The method of claim 18 wherein said step of coupling said automatic injector to said rigid structure comprises aligning said output of said automatic injector to be normal to the skin of the living being

24. The method of claim 18 wherein said output comprises a needle and wherein said step of coupling said automatic injector to said rigid structure comprises aligning said needle of said automatic injector to be normal to the skin of the living being

25. The method of claim 24 wherein said needle passes through said aperture and into the skin of the living being for a predetermined period of time and wherein said predetermined period of time comprises at least 10 minutes.

26. An apparatus for automatically delivering a solution to a living being, said apparatus comprising:
an automatic injector having a housing with a proximal and distal end, said proximal end comprising a skin-engaging surface and a hole for permitting the solution to pass therethrough;
said skin-engaging surface comprising releasable coupling means for releasably coupling said apparatus to the living being; and
wherein said apparatus delivers the solution, when activated, through the skin of the living being without the need for a person to hold said apparatus during solution delivery.

27. The apparatus of claim 26 wherein said apparatus comprises a longitudinal axis and wherein said living being-engaging surface is oriented perpendicular to said longitudinal axis.

28. The apparatus of claim 26 wherein said releasable coupling means comprises an adhesive means coupled to said living being engaging surface.

29. The apparatus of claim 28 wherein said adhesive means comprises:
a first layer comprising a first size and having a first pair of opposing surfaces and wherein one of said first pair of opposing surfaces is coupled to said living being engaging member; and
a second layer comprising a second size larger than said first size and including a second pair of opposing surfaces, wherein one of said second pair of opposing surfaces is coupled to the other one of said first pair of opposing surfaces and wherein the other one of said second pair of opposing sides comprises a biocompatible adhesive.

30. The apparatus of claim 29 wherein said one said first pair of opposing surfaces comprises an adhesive such that said first layer is adhesively secured to said living being-engaging member.

31. The apparatus of claim 30 wherein the other one of said first pair of opposing surfaces also comprises an adhesive such that said one of said second pair of opposing surfaces is also adhesively coupled to the other one of said first pair of opposing surfaces.

32. The apparatus of claim 26 wherein said predetermined period of time comprises at least 10 minutes.

33. The apparatus of claim 28 further comprising:
a release liner that covers said adhesive until said apparatus is ready for use; and
a cover that is releasable from said proximal end of said apparatus, said release sheet having a portion thereof engaged with said cover and whenever said cover is removed from said proximal end before use, said release liner is simultaneously removed.

34. The apparatus of claim 33 further comprising:
a needle that passes through said hole and into the skin when activated to deliver the solution; and
a removable needle protector engaged with said needle before use and wherein said cover comprises latch members that engage said needle protector, said needle protector being simultaneously removed from said needle when said cover is removed.

35. The apparatus of claim 34 wherein said needle passes through said hole and into the skin of the living being for a predetermined period of time and wherein said predetermined period of time comprises at least 10 minutes.

36. A method for automatically injecting a solution into a living being, said method comprising the steps of:
coupling a proximal end of an automatic injector to the skin of the living being, said automatic injector having an aperture in said proximal end that is coupled to the skin of the living being; and
activating said automatic injector to cause the solution to pass through said aperture and into the skin and without the need for a person to hold said automatic injector during solution delivery.

37. The method of claim 36 wherein said step of coupling said proximal end of said automatic injector to the skin of the living being comprises:
applying an adhesive to said proximal end of said automatic injector that eventually is in contact with the skin of the living being;
covering said adhesive with a release liner;
wiping the skin of the living being where said automatic injector is to be coupled;
removing said release liner from said portion of said proximal end when ready for use; and
applying firmly said portion of said proximal end to the skin of the living being.

38. The method of claim 37 wherein said step of applying an adhesive to said portion of a rigid structure comprises:
coupling one surface of a first pair of opposing surfaces of a first layer, having a first size, to said portion of said rigid structure;
coupling one surface of a second pair of opposing surfaces of a second layer having a second size, larger than said first size, to the other one of said first pair of opposing surfaces; and
covering the other one of said second pair of opposing surfaces with a biocompatible adhesive.

39. The method of claim 38 wherein said step of coupling one surface of a first pair of opposing surfaces of a first layer to said portion of said rigid structure comprises applying an adhesive to said one surface of said first pair of opposing surfaces.

40. The method of claim 39 wherein said step of coupling one surface of a second pair of opposing surfaces of a second layer having a second size, to the other one of said first pair of opposing surfaces comprises applying an adhesive to the other one of said first pair of opposing surfaces.

41. The method of claim 36 wherein said activating said automatic injector to cause the solution to pass through said aperture and into the skin comprises passing said solution into the skin in a direction normal to the skin.

42. The method of claim 36 wherein said automatic injector comprises a needle that passes through said aperture and wherein said step of coupling said proximal end of said automatic injector to the skin of the living being comprises aligning said needle of said automatic injector to be normal to the skin of the living being

43. The method of claim 42 wherein said needle passes through said hole into the skin of the living being for a predetermined period of time and wherein said predetermined period of time comprises at least 10 minutes.
